(19) United States
(12) Patent Application Publication
(10) Pub. No.: US 2017/0143623 A1
(43) Pub. Date: May 25, 2017

(54) ORODISPERSIBLE FILMS HAVING QUICK DISSOLUTION TIMES FOR THERAPEUTIC AND FOOD USE

(71) Applicant: Pharmafilm S.r.l., Gaggiano (MI) (IT)

(72) Inventors: Francesco Cilurzo, Gaggiano (MI) (IT); Maurizio Di Grigoli, Gaggiano (MI) (IT); Paola Minghetti, Gaggiano (MI) (IT); Stefania Pagani, Gaggiano (MI) (IT)

(21) Appl. No.: 15/423,887

(22) Filed: Feb. 3, 2017

Related U.S. Application Data
(63) Continuation-in-part of application No. 14/430,255, filed on Mar. 23, 2015.

(30) Foreign Application Priority Data
Sep. 28, 2012 (IT) MI2012A001628

(51) Int. Cl. A61K 9/00; A61K 47/36; A61K 47/32; A61K 31/196; B65B 63/08; A61K 31/4985; A61K 31/57; A61K 31/568; A23L 33/10; A61K 9/70; A61K 31/519

(52) U.S. Cl. CPC A61K 9/0056; A61K 9/7007; A61K 47/36; A61K 47/32; A61K 31/196; A61K 31/519; A61K 31/4985; A61K 31/57; A61K 31/568; A23L 33/10; B65B 63/08; A23V 2002/00

(57) ABSTRACT

The present invention concerns an orodispersible self-supporting film free from hydrocolloids comprising:
a) a film-forming substance consisting of a maltodextrin in an amount comprised between 40 and 80% by weight;
b) one or more plasticizer in a total amount comprised between 15 and 55% by weight;
e) a surfactant System in an amount comprised between 0.5 and 6% by weight;
d) an active ingredient for food or therapeutic use in an amount between 0.05 and 30% by weight,
said orodispersible self-supporting film free from hydrocolloids further containing a homopolymer or a copolymer of vinyl acetate in a quantity comprised between 1 and 20% by weight where the percentages are calculated on the total weight of said film.

This U.S. Non-Provisional application is a Continuation-in-Part of U.S. Ser. No. 14/430,255 filed on Mar. 23, 2015, which is a U.S. National Stage Application of PCT/IB2013/058882 filed on 26 Sep. 2013, which claims priority to and the benefit of Italian Application No. MI2012A001628, filed on 28 Sep. 2012, the contents of which are incorporated herein by reference in their entireties.

### FIELD OF THE INVENTION

The present invention concerns orodispersible self supporting films having quick dissolution time for therapeutic and food use.

### STATE OF THE ART

Orodispersible self supporting films for releasing active ingredients for therapeutic or food use have been known for a long time and are available on the market.

These films disintegrate quickly in the mouth releasing the active ingredient.

Many of the films known at the state of the art use pullulan as the film forming component which is, however, an ingredient that is expensive and difficult to find.

It has thus been attempted to replace pullulan with less expensive ingredients that are, in any case, capable of maintaining the properties like their quick dissolution times, mouth freshness, marked aroma, and simplicity of preparation.

Chapdelaine et al. (WO 2003011259) described a film having quick dissolution times containing maltodextrin and hydrocolloids, in quantities that are greater than 10%, as film forming component. Hydrocolloids were necessary in order to facilitate the disintegration of the film but did not give the sensation of having a clean mouth since they tend to gel in contact with saliva.

Cilurzo et al. (US 2009/0017085) described self-supporting films for releasing active ingredients for therapeutic or food use based on maltodextrin and a plasticizer, totally without hydrocolloids. These films quickly disintegrated in the mouth and released the active ingredient in the oral cavity keeping the sensation of having a clean mouth that is indeed of pullulan-based films. Films composed by maltodextrin and plasticizers free from hydrocolloids had a drawback concerning their physical stability as they tended to become brittle over time and were not suitable to be handled during manufacturing process

Ibrahim et al. (US 2005/0281757) described a composition for delivery of an oral care substance to a dental surface upon application of the composition thereto. The composition was a flexible film comprising the oral care substance dispersed in a film-forming effective amount of a polymeric matrix having a hydrophilic component and a hydrophobic component in a weight ratio selected such that the film is substantially dissolvable in saliva in a period of time effective for delivery of the oral care substance [claim 1]. Adjusting the weight ratio of the more hydrophilic vinylpyrrolidone and the more hydrophobic vinylacetate monomers enables dissolution time to be controlled for optimum delivery of the oral care substance. The film described by Ibrahim were substantially dissolvable in saliva in about 5 to about 60 minutes [claim 13]. This dissolution time did not comply with specification of orodispersible films, since it is generally recognized that an orodispersible dosage form should disintegrate in less than 3 min as the PhEur states.

### SUMMARY OF THE INVENTION

The aim of this work was the development of a film based on maltodextrin, a not expensive polymer, easy to find, free from hydrocolloids. These films were characterized by quick disintegration time (less then 3 minutes), and suitable mechanical properties in order to be handled during manufacturing process.

It has now been surprisingly found that it is possible to avoid brittleness of the films based on maltodextrin and plasticizer by incorporating, in the composition, a homopolymer or copolymer of vinyl acetate, preferably polyvinil acetate (PVA) or polyvinylpirrolidone vinyl acetate (PVP-VA).

The present invention concerns orodispersible self-supporting films without hydrocolloids comprising:

a) a film-forming substance consisting of a maltodextrin in a quantity comprised between 40 and 80% by weight;

b) one or more plasticizer in a quantity comprised between 15 and 55% by weight;

e) a surfactant system in a percentage comprised between 0.5 and 6% by weight;

d) an active ingredient for food or therapeutic use in a quantity comprised between 0.05 and 30% by weight,

said orodispersible self-supporting film free from hydrocolloids further containing a homopolymer or a copolymer of vinyl acetate in a quantity comprised between 1 and 20% by weight, where the percentages are calculated on the total weight of said film.

The polymers in general of vinyl acetate and in particular polyvinyl acetate are insoluble in water, the latter being used in many medicinal products, for example in pharmaceutical formulations with a prolonged release over time, or as a base in chewing gum.

The applications of PVP-VA rely mainly on its good binding properties, its affinity to hydrophilic and hydrophobic surface and its relatively low hygroscopicity. Because of these properties, PVP-VA is used as a binder in the production of granules and tablets by wet granulation, as a dry binder in direct compression, as a protective layer and subcoat for tablets cores, as a film forming agent in sprays and as a matrix.

The films, object of the present invention, have a quick dissolution time: addition of the more hydrophilic PVP-VA or the more hydrophobic PVA does not affect dissolution time of films.

### BRIEF DESCRIPTION OF THE DRAWINGS

FIG. 1 shows the comparative in vitro dissolution profile of sildenafil at pH 5.5.

FIG. 2 shows the sildenafil pharmacokinetic profiles of the two investigational medicinal products.

FIG. 3 shows the dissolution profiles of Tadalafil 20 mg Orodispersible films and reference product Cialis®.

### DETAILED DESCRIPTION OF THE INVENTION

The orodispersible films of the invention have disintegration times, evaluated in vitro and in vivo, that are lower than 3 minutes, they do not stick, they do not expand and are stable over time as far as the mechanical properties of elasticity and tensile strength are concerned, even when exposed to air.

The homopolymer or copolymer of vinyl acetate is in a quantity comprised between 1 and 20% by weight.

In particular the homopolymer of vinyl acetate (i.e. polyvinyl acetate—PVA) used in the invention has preferably an average molecular weight of between 5000 and 500000, more preferably between 250000 and 450000. A polyvinyl acetate that can be used in the invention is that sold with trademark Kollicoat® SR 30D commercialised by BASF.

The copolymer of vinyl acetate is preferably PVP-VA and a commercially material that can be used is Kollidon VA 64E with a ratio PVP: VA of 60:40.

Both polymers (PVA and PVP-VA 60:40) fulfil the monographs in the current versions of the European Pharmacopoeia, United States Pharmacopoeia and Japanese Pharmaceutical Excipients, and can be used for pharmaceutical product.

Preferably, the content of PVAc or PVP-VA in the film according to the present invention is between 2 and 10%, more preferably between 2.5 and 10%, even more preferably between 3 and 10%. According to a particularly preferred solutions the content of PVAc or PVP-VA in the film is between 3 and 6% or 3 and 5.5% by weight on the total weight of the the content of PVAc or PVP-VA in the film.

The maltodextrin used in the self-supporting film of the present invention has preferably a dextrose content, expressed in equivalents, that is less than 50, and more preferably is between 5 and 40.

The plasticizer used in the film of the present invention is preferably selected from the group consisting of water, polyalcohols, esters of citric acid, sebacic acid esters or mixtures thereof.

Particularly preferred are water, propylene glycol, glycerine, mannitol, sorbitol, maltitol and mixtures thereof.

The use of water as plasticizer is well known to the skilled in the art (Water Science Reviews, volume 3, Publisher: Cambridge University Press, Editors: Felix Franks, pp. 79-185).

The surfactant system used in the film of the present invention consists of one or more surfactants, preferably selected from the group consisting of sorbitan derivatives, sorbitol derivatives, esters of sucrose, fatty acid esters and their mixtures.

The active ingredient for food use is preferably an active ingredient with a breath freshening action and/or suitable for oral hygiene, preferably eugenol or menthol or a vegetal extract or an active ingredient of natural origin, suitable for nutritional supplementation, preferably mineral salts among those normally used for such a purpose or one or more vitamins.

The active ingredient for therapeutic use can be an ingredient with essentially topical action on the oral cavity selected from: antibacterial, antifungal, antiviral agents or disinfectants of the oral cavity; or it can be an ingredient with an essentially systemic action selected from the group of: anti-inflammatory, analgesic, antipsychotic, hypnotic, anxiolytic, muscle relaxant, antimigraine, antiparkinsonian, antiemetic, antihistaminic, beta blocker, anti-asthmatic anti-hypertensive, antitussive, laxative agents, inhibitors of type V phosphodiesterase, antikinetosis agents and hormones.

Active ingredients contained in such films are preferably selected from the group consisting of: Piroxicam, Ketoprofen, Diclofenac, Tramadol, Morphine, Nifedipine, Diazepam, Lorazepam, Alprazoiam, Bromazepam, Triazolam, Lormetazolam, Zolpidem, Paracetamol, Selegiline, Atenolol, Salbutamol, Sumatriptan, Clozapine, Ceterizine and their pharmaceutically acceptable salts.

A preferred active ingredient is an inhibitor of type V phosphodiesterase selected from sildenafil citrate and tadalafil or a hormone selected from progesterone and testosterone.

Moreover, the films according to the invention can possibly contain other excipients selected in the class of non-stick substances like for example colloidal silica or talc, sweeteners, flavourings, colorants, preservatives, buffer systems or mixtures thereof.

The films, object of the invention, can be manufactured with known processes, like those described in EP 1689374 by Cilurzo et al.

In particular, a process can be used comprising the steps of:

i) dispersing the maltodextrin, the plasticizer, the surfactant system, the homopolymer or copolymer of vinyl acetate and the active ingredient for therapeutic or food use in a polar solvent;

ii) laminating the mixture obtained in the previous step on a release liner;

iii) drying in the oven at a temperature preferably comprised between 50 and 140° C.;

iv) removing the release liner from the film obtained in the preceding step;

v) cutting the film with the desired dimensions and packaging them.

The polar solvent used in step i) is preferably selected from water, water-mixable solvents or relative mixtures. According to a particularly preferred solution it consists of water;

### Example 1—Preparation of Placebo Orodispersible Films

The polymer mixture used for preparing the films was obtained by solubilizing maltodextrin DE 6 in a suitable amount of water kept at T=80° C.

Subsequently The mixture was gradually cooled and glycerine, the surfactants, the homopolymer of vinyl acetate and the other components were added in the ratios indicated in Table 1. The system obtained is kept under stirring until all the components were dissolved.

The composition of the polymer mixtures used for preparing the film is shown in Table 1.

TABLE 1
Dry composition % (w/w)

| | Dry composition %, w/w | | | | | |
|---|---|---|---|---|---|---|
| Formulation | F1 | F2 | F3 | F4 | F5 | F6 |
| MDX DE6 | 78.99 | 80.72 | 76.50 | 74.87 | 70.78 | 62.68 |
| Glycerin | 18.05 | 14.57 | 17.48 | 17.11 | 16.17 | 14.32 |
| Span80 | 2.96 | 3.71 | 3.01 | 3.01 | 3.05 | 2.99 |
| PVAc | — | 1.00 | 3.01 | 5.01 | 10.00 | 20.00 |

The preparation of the film was carried out using the Mathis Labcoater-Labdryer model LTE—S (M) (CH) according to a method that foresees coating the mixture on a protective silicone sheet. The operation conditions used are as follows:
Coating speed: 1 m/min
Drying time: 15 min
Drying temperature: 60° C.
Rotation speed of the fan: 1800 rpm (revs/minute)
Coating thickness: 380 μm

The films thus prepared were separated by the protective sheet, cut with the desired dimensions and preserved in waterproof and lightproof packets.

### Example 2—Determination of Tensile Properties

The analysis of the tensile properties was carried out in accordance with ASTM standards (International Test Method for Thin Plastic Sheeting) (D 8 82-02) using an Acquati electronic dynamometer mod. AG/MC1 (I) on which a load cell of 5 N was assembled. The result of the tests is expressed as an average of the analysis on 5 samples for each formulation. The film was preliminarily cut into strips with a length of 100 mm and width of 12.5 mm Once it was verified that there were no breaks or a lack of homogeneity in the matrix, the samples were positioned longitudinally between two pneumatic clamps spaced at 60 mm from one another. The separation velocity of the clamps was set at 500 mm/min. The test was considered finished once the film broke. Variations in the rigidity of the material were measured by determining the elastic modulus (EM) after the preparation of the films and after three months of preservation at 40° C.

The addition of PVAc was considered positive if the variation of this value (EM) after 3 months from the preparation was lower than the EM variation of the formulation free of PVAc.

Results

The elastic modulus values EM are shown in Table 2.

TABLE 2
Elastic modulus values of formulations F1-F6 after preparation
thereof and after being preserved for three months at 40° C.
and variation percentage (V) thereof over time.

Elastic modulus (EM)

| Formulation | 0 months | 3 months | (V) |
|---|---|---|---|
| F1 | 26 | 61 | 135% |
| F2 | 59 | 78 | 32% |
| F3 | 113 | 119 | 5% |
| F4 | 55 | 69 | 25% |
| F5 | 35 | 40 | 14% |
| F6 | 14 | 23 | 64% |

The results show how the addition of PVA in the range 1-20% makes it possible to improve the mechanical properties of the film. Indeed, with respect to the reference formulation 1, the addition of PVA in the selected range makes it possible to reduce the variation (V) of elastic modulus parameter EM over time.

### Example 3—Preparation and Characterization of Placebo Orodispersible Films Containing Copolymer of Vinyl Acetate

PVP-VA (60:40) was selected as copolymer of vinylacetate, to investigate the effect on tensile properties of films. Formulations reported in the Table 3 were prepared according to the method described in the Example 1.

TABLE 3
Formulations of film containing PVP-VA

| | Dry composition w/w % | | | | | | | | | |
|---|---|---|---|---|---|---|---|---|---|---|
| COMPONENTS | F7 | F8 | F9 | F10 | F11 | F12 | F13 | F14 | F15 | F16 |
| Maltodextrin DE6 | 78.5 | 77.5 | 76.5 | 74.5 | 72.5 | 70.5 | 68.5 | 79.0 | 76.5 | 75.0 |
| Glycerin | 20.0 | 20.0 | 20.0 | 20.0 | 20.0 | 20.0 | 20.0 | 18.0 | 17.5 | 17.0 |
| Tween 80 | 0.57 | 0.57 | 0.57 | 0.57 | 0.57 | 0.57 | 0.57 | — | — | |
| Peceol | 0.93 | 0.93 | 0.93 | 0.93 | 0.93 | 0.93 | 0.93 | — | — | |
| Span 80 | — | — | — | — | — | — | — | 3.0 | 3.0 | 3.0 |
| PVP/VA | — | 1.0 | 2.0 | 4.0 | 6.0 | 8.0 | 10.0 | | 3.0 | 5.0 |

Characterization of Placebo Orodispersible Films

Water content, disintegration time and tensile properties of films after preparation (T0) and after air exposure were evaluated.

Water Content

The water content was determined gravimetrically after keeping films samples of 9 cm² surface at the temperature of 130° C. over a 2 h period. The results were expressed as the mean of three determination according to the following equation:

$$W \% = \frac{Wo - Wf}{Wo} \times 100$$

Where Wo and Wf are the initial and the final weight, respectively.

Disintegration Test

Disintegration test was performed according to the specifications of orodispersible tablet reported in Ph. Eur. 5.4 ed. (2.9.1) by using samples of 9 cm².

Tensile Properties

Mechanical properties of orodispersible films (F0-F5) were evaluated using a texture analyzer Tinus Olsen H5K-T equipped with a 100 N load cell. Each film was placed in tensile grips on the texture analyzer. Initial grip separation was 100 mm and crosshead speed was 50 mm/min. The test was considered concluded at the film break. Measurements were run in five replicates for each film. Tensile strength, elongation at break and elastic modulus were calculated to evaluate tensile properties of orodispersible films, as recommended by the ASTM D882-02 Standard Test method.

Tensile strength (TS): was calculated by dividing the maximum load by the original cross sectional area of the specimen, it was expressed in force per unit area (MPa).

Percent elongation at break (E %): was calculated by dividing the extension at the moment of rupture of the specimen by the initial gage length of the specimen and multiplying by 100.

Elastic modulus or Young's modulus (EM) was calculated as the slope of the linear portion of the stress strain curve. The results was expressed in force per unit area (MPa).

Before the test, film thickness of each specimen was measured by using an electronic micrometer (ChemInstruments, USA).

Results

Formulations having the same qualitative composition, i.e. F7-F13 and F14-F16, were compared to each other. All films disintegrated in less than 1 minute evidencing that the addition of PVP-VA in different ratios did not affect the dissolution of MDX films.

Tensile properties of orodispersible films gives an indication of the strength and elasticity of the film, reflected by the parameters: tensile strength (TS), elongation at break (E %) and Elastic modulus or Young modulus (EM). The ductility of films is expressed as elongation at break (E %), while the toughness is expressed as the tensile strength and elastic modulus.

The flexibility and toughness of the film affect manufacturing process of films such as cutting, film formation and packaging.

Oral film should be flexible to be handled without failure and at the same time should exhibit a tensile strength that guarantees a suitable toughness to allow films to be self-supporting. Elongation at break should be low to avoid deformation of films during manufacturing process.

Tables 4 and 5 show results of water content and tensile properties of films F7-F13 immediately after preparation on lab-scale, minimizing air exposure, and after air exposure (to simulate the usual environment on large-scale production).

TABLE 4
Tensile properties of packaged films containing PVP-VA,
peceol and tween 80 immediately after preparation.

| | | Water | Tensile | | E |
|---|---|---|---|---|---|
| | Content of | content | Strength | Elongation | Modulus |
| Formulation | PVP-VA (%) | (%) | (Mpa) | at break (%) | (Mpa) |
| F7 | 0 | 8.2 | 0.63 | 61.4 | 56.4 |
| F8 | 1 | 10.1 | 0.44 | 40.0 | 41.3 |
| F9 | 2 | 10.1 | 1.29 | 48.0 | 95.8 |
| F10 | 4 | 9.4 | 1.17 | 33.9 | 98.1 |
| F11 | 6 | 9.5 | 1.42 | 34.9 | 116.0 |
| F12 | 8 | 9.5 | 1.74 | 28.0 | 154.0 |
| F13 | 10 | 9.0 | 2.81 | 12.0 | 216.0 |

Films loaded with PVP-VA were homogeneous and opaque. The residual water content was in the range 8.2-10.1%. All films were handled without failure. Increase of PVP-VA amount in the formulations caused increase of tensile strength and elastic modulus and decrease of elongation at break. Film with more PVP-VA were tougher, stiffer and less ductile. PVP-VA affected tensile properties of films.

TABLE 5
Tensile properties of film containing PVP-VA after air exposure

| | Content of | | Tensile | Elongation | E |
|---|---|---|---|---|---|
| | PVP-VA | Water | Strength | at break | Modulus |
| Formulation | (%) | content (%) | (MPa) | (%) | (Mpa) |
| F7 | 0 | 7.2 | n.a | n.a | n.a |
| F8 | 1 | 8.7 | 1.17 | 1.28 | 212 |
| F9 | 2 | 8.5 | 2.26 | 0.92 | 316 |
| F10 | 4 | 8.0 | 2.54 | 1.26 | 299 |
| F11 | 6 | 8.2 | 2.47 | 0.91 | 346 |
| F12 | 8 | 8.2 | 2.82 | 1.02 | 430 |
| F13 | 10 | 7.6 | 3.35 | 6.10 | 282 |

As stated above, after air exposure, orodispersible films F7, without vinyl acetate polymers, were not tested because films were very brittle and were subjected to failures during handling. Formulations F8-F13 with PVP-VA in the range 1-10% were tough and flexible and could be handled without failure.

Formulations F8-F13 are suitable to be handled during the different steps of the manufacturing process, because can be exposed to air without any failure, while formulation F7, without PVP-VA, is air-sensitive and cannot be handled in a normal production environment.

Tables 6 and 7 show results of tensile properties of films F14-F16 immediately after preparation on lab-scale, minimizing air exposure, and after air exposure (to simulate the usual environment on large-scale production).

TABLE 6
Tensile properties of F14-F16

| | Content of | | | |
|---|---|---|---|---|
| | PVP-VA | Tensile Strength | Elongation at | E Modulus |
| Formulation | (%) | (MPa) | break (%) | (MPa) |
| F14 | 0 | 0.45 | 24.2 | 23.3 |
| F15 | 3 | 0.53 | 29.2 | 24.3 |
| F16 | 5 | 1.16 | 17.2 | 75.7 |

TABLE 7
Tensile properties of F12, F13 and F14 after air exposure

| | Content of | | | |
|---|---|---|---|---|
| | PVP-VA | Tensile Strength | Elongation at | E Modulus |
| Formulation | (%) | (MPa) | break (%) | (MPa) |
| F14 | 0 | — | — | — |
| F15 | 3 | 2.04 | 13.8 | 179 |
| F16 | 5 | 2.18 | 4.81 | 198 |

Also in this case, after air exposure, orodispersible films F14 without any vinyl acetate polymer were not tested because films were very brittle and were subjected to failures during handling. Formulations 15 and F16 with PVP-VA at 3 and 5% respectively became tougher, but were enough flexible to be handled without failure Formulations F15 and F16 are suitable to be handled during the different steps of the manufacturing process, because can be exposed to air without any failure, while formulation F14, without PVP-VA, is air-sensitive and cannot be handled in a normal production environment, like F7.

### Example 4—Preparation and Characterization of Orodispersible Films Containing Diclofenac

Preparation of the Film

The films, the composition of which is shown in Table 8, were prepared as described in Example 1

TABLE 8
Composition and technological characteristics of the films

Formulations w/w %

| Composition | D1 | D2 |
|---|---|---|
| Maltodextrins DE6 | 57.11 | 60.73 |
| Glycerin | 3.80 | 4.04 |
| Span 80 | 1.44 | 1.53 |
| PVAc | 5.00 | — |
| Sorbitol | 6.66 | 7.08 |
| Peach aroma | 3.42 | 3.64 |
| Betaine | 2.78 | 2.95 |
| Mint flavour | 1.90 | 2.02 |
| Sucralose | 1.14 | 1.21 |
| Tween 20 | 0.72 | 0.77 |
| Titanium dioxide | 0.23 | 0.24 |
| Diclofenac epolamine | 15.80 | 15.78 |

Determination of the Mechanical Properties

The elastic modulus (EM) was determined as described in Example 1.

Disintegration Test

The disintegration test was carried out according to the specifications for orodispersible tablets shown in Eur. Ph. Ed. 7.0, setting the time T<3 min and using samples of 6 cm².

For every formulation, three tests were carried out and the results were expressed as an average±standard deviation.

Dissolution Test

The dissolution test in vitro was carried out on samples of 6 cm² using “Basket Dissolution Apparatus” (Eur. Ph. 7.0, Section 2.9.3).

The following parameters characterise the method used for evaluating the % drug dissolved:

Equipment: Sotax AT7 Smart Dissolution system with Basket

Temperature: 37±0.5° C.

Dissolution medium: phosphate buffer pH 6.8

Volume of dissolution: 500 mL

Rotation speed: 100 rpm (revs/minute)

Sampling time: 5 minutes

The buffer volume described was inserted in the 7 vessels of the dissolution system and the system was left to settle at the set temperature of 37° C. A film was introduced in each of the first 6 baskets, the 7^{th} vessel was used as the control and therefore the relative basket was kept empty.

Once the set temperature was reached, the baskets were lowered into the dissolution medium. After 5 minutes, an aliquot was taken from each vessel. The samples obtained were analysed in HPLC by using the following method.

HPLC Agilent 1100, with Grace Alltima HP C18 column with dimensions 100×4.6 mm and 3 μm. An isocratic elution was carried out comprising mixing a mobile phase A and a mobile phase B. Phase A consisted in 90% of a 20 mM phosphate buffer at pH 2.0 prepared dissolving 3.12 g of sodium dihydrogen phosphate in 1 litre of Milli-Q water and regulating the pH to 2.0 with conc. phosphoric acid (H₃PO₄) and 10% of tetrahydrofuran for HPLC. Phase B consisted of grade HPLC Methanol. The two phases were mixed in the proportions indicated here: Phase A: 40%, Phase B: 60%.

The column temperature was set at 40° C., flow 1.3 ml/min, selected wavelength 254 nm, injection volume 2 μl.

Results

The results shown in table 9 indicate that the addition of PVAc makes it possible to obtain films having mechanical properties and tensile strength that are considerably higher than reference films, without PVAc, while maintaining unaltered disgregation and release characteristics.

TABLE 9
Characterization of the films containing Diclofenac

Results

| Test | D1 | D2 |
|---|---|---|
| Mechanical properties EM (kPa) | 160 | 118 |
| Dissolution test | 98.6 | 104.5 |
| % dissolved (limit >80%) | | |
| Disintegration test | Complies | Complies |
| T <3 minutes | | |

### Example 5—Sildenafil Orodispersible Film: Preparation, In-Vitro and In-Vivo Bioequivalence Study

A sildenafil mass was prepared as described below using the amounts of the components reported in Table 11

Glycerol, polysorbate 20, propylene glycol monocaprylate and water were added into the mixer and stirred at 25° C. Titanium dioxide and subsequently sildenafil citrate were added while mixing. Then polyvinyl acetate dispersion was added to the mixture under stirring. Afterward maltodextrin and a solution containing colour and flavours were added. The mixture was stirred until the blend was blue and homogeneous.

The mixture was spread on a silicone/PET liner and dried in a oven. The film, removed from the liner, is carefully cut into strips and pouched in suitable sealed sachets.

The composition of the film is reported in the following Table 11.

TABLE 11
Composition the film of sildenafil film

| | Quantitative formula |
|---|---|
| Components | of drug product (mg) |
| Sildenafil Citrate | 140.4 |
| (corresponding to Sildenafil) | (100.0) |
| Maltodextrin | 232.3 |
| Sucralose | 3.0 |
| Lemon flavor | 10.0 |
| Grapefruit flavour | 10.0 |
| Polyvinyl Acetate dispersion 30% (PVAc) | 24.2 |
| Titanium dioxide | 0.5 |
| Indigotine E132 | 0.1 |
| Polysorbate 20 | 1.7 |
| Propilene glycolmonocaprylate (type II) | 6.5 |
| Glycerol | 55.6 |
| Purified water | 52.3 |
| Total | 536.6 |

Bioequivalence Study

In-Vitro Study: Comparative In Vitro Dissolution Profile at pH 5.5

The dissolution profile of the sildenafil 100 mg orodispersible film obtained above was compared to that of the conventional marketed 100 mg film-coated tablet (Viagra®, Pfizer)

Dissolution Conditions:

| | |
|---|---|
| Samples | n = 12 of reference product Viagra ® |
| | n = 12 of test product Sildenafil orodispersible films |
| Apparatus | Basket apparatus (Ph. Eur. Method 2.9.3, Apparatus 1) |
| Medium | Buffer solution pH 5.5 (Ph. Eur. 4002000) |
| | Dissolve 13.61 of potassium dihydrogen phosphate in |
| | water and dilute to 1000.0 mL with the same solvent |
| | (solution A). Dissolve 35.81 g of disodium hydrogen |
| | phosphate in water and dilute to 1000.0 mL with the |
| | same solvent (solution B). Mix 96.4 mL of solution A |
| | and 3.6 of solution B. |
| Temperature | 37° C. ± 0.5° C. |
| Stirring speed | 150 rpm |
| Medium | 900 ml |
| Volume | |
| Sampling time | 5, 10, 15, 30 minutes |

Results:

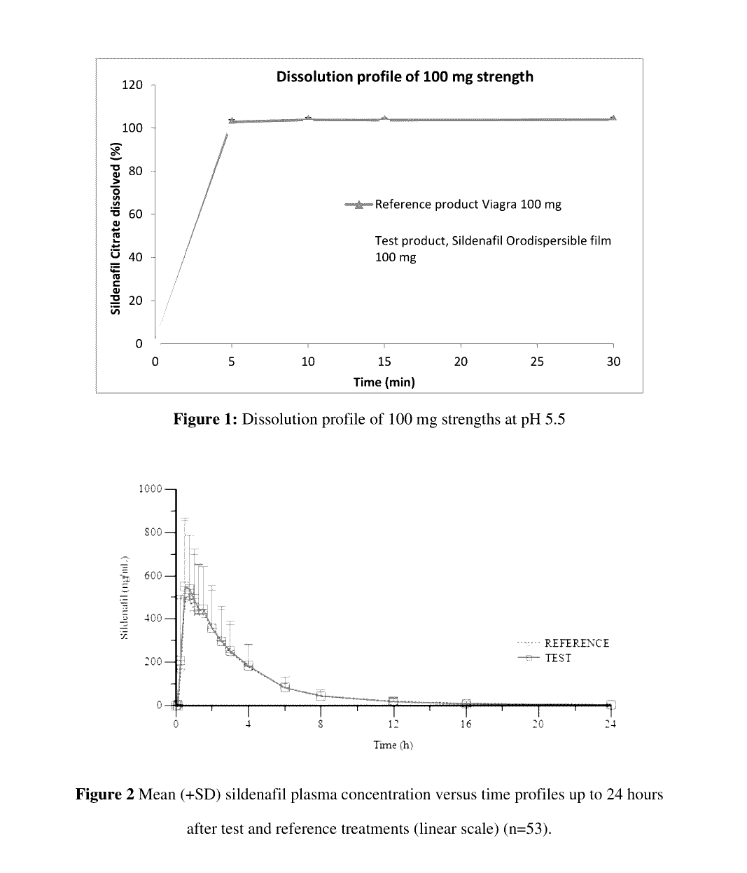

The results are reported in FIG. 1

Dissolution profiles of the Sildenafil Orodispersible film and the Viagra® tablet (reference drug) are overlapping.

More than 85% of the label content dissolved within 15 minutes, so both the drug products, Viagra® tablet and Sildenafil Orodispersible film, are considered “very rapidly” dissolving, and the similarity of their dissolution profiles is accepted without any mathematical calculation, as stated in the Guideline on the investigation of Bioequivalence.

Results of the comparative in vitro dissolution reflects the bioequivalence of Sildenafil Orodispersible films to the reference product Viagra® tablet.

In Vivo Study

The pharmacokinetics of the sildenafil 100 mg orodispersible film was compared to that of the conventional marketed 100 mg film-coated tablet (Viagra®, Pfizer) after single dose administration to 53 healthy male volunteers (aged 18-51 years) in a randomized, open, 2-way cross-over bioequivalence study. Each subject received a single oral dose of 100 mg of sildenafil as test or reference formulation and then blood samples for pharmacokinetic analysis were collected up to 24 hours post-dosing.

The Sildenafil 100 mg orodispersible film test treatment was administered to the subject without water, while the sildenafil 100 mg reference formulation was administered to the subject with 240 mL of water to help the swallowing.

Rate (peak plasma concentration; Cₘₐₓ), extent (area under the curve from administration to last observed concentration time; AUC₀₋ₜ) of sildenafil absorption and plasma pharmacokinetic profiles of sildenafil after single dose administration of test and reference were compared.

Results

The majority of the subjects judged the palatability of the sildenafil 100 mg orodispersible film to be good/acceptable. The dissolution in the mouth took always less than 3 minutes, without use of water, for all the subject.

Sildenafil administered as a single dose of test or reference product was well tolerated.

The mean±standard deviation (SD) plasma sildenafil pharmacokinetic parameters are reported in Table 12.

TABLE 11
Mean Plasma Sildenafil Pharmacokinetic Parameters (n = 53)

| | | | Point |
|---|---|---|---|
| Parameter | Test | Reference | estimate (%) |
| Cₘₐₓ (ng/mL) | 645.30 ± 281.83 | 664.96 ± 317.91 | 99.53 |
| AUC₀₋ₜ | 1971.10 ± 978.16 | 1900.25 ± 957.31 | 105.00 |
| (ng/mL × h) | | | |
| AUC_{0-∞} | 2001.10 ± 1008.96 | 1932.13 ± 987.70 | 104.79 |
| (ng/mL × h) | | | |
| tₘₐₓ (h) | 0.75 (0.50-3.00) | 0.75 (0.25-2.50) | — |

Notes:
Values are mean ± standard deviation (SD), except for tₘₐₓ, median (range). Point estimate, test/reference ratio of geometric means.
Test = sildenafil 100 mg orodispersible film; Reference = Viagra ® 100 mg film-coated tablet.

The mean sildenafil plasma concentration-time profiles up to 24 hours after single-dose administration of sildenafil 100 mg orodispersible film and film-coated tablet were nearly superimposable. The bioequivalence test was fully satisfied for sildenafil in terms of rate and extent of bioavailability. The results suggest that the new orodispersible film formulation can be used interchangeably with the conventional film-coated formulation.

FIG. 2 shows the sildenafil pharmacokinetic profiles of the two investigational medicinal products (Test=sildenafil 100 mg orodispersible film; Reference=Viagra® 100 mg film-coated tablet) up to 24 hours after sildenafil 100 mg test and reference treatments.

The mean plasma concentration-time profiles up to 24 hours of sildenafil 100 mg orodispersible film and the film-coated tablet were nearly superimposable.

The sildenafil 100 mg orodispersible film (test) and the 100 mg film-coated tablet (reference) were determined to be bioequivalent with respect to sildenafil rate and extent of absorption.

Moreover, Sildenafil orodispersible film offers a convenient, discrete method of intake with a rapid onset of action and the added patient convenience and acceptability of a dosage form that does not require administration with water, of particular benefit for men who have difficulty with swallowing conventional tablet or in whom daily fluid intake is restricted, as the orodispersible film does not require administration with water.

The Sildenafil orodispersible film has the advantages of an orodispersible formulation that dissolved rapidly in the oral cavity, without drinking or chewing.

The pharmacokinetics study show that the Sildenafil 100 mg orodispersible film formulation and the 100 mg film-coated tablet were bioequivalent, without statistically significant difference. The results suggest that the new orodispersible film formulation could represent a valid alternative to the current marketed products for the treatment of erectile dysfunction, with the same efficacy but a better patient's compliance.

### Example 6: Tadalafil Orodispersible Films: Formulation and Comparative Dissolution Study with Reference Product

An orodispersible film containing Tadalafil was prepared according to the procedure reported in Example 5, using the components and the amounts reported in the following Table 13.

TABLE 13
Composition of Tadalafil Orodispersible film

| Compositions | Dry composition w/w % | Dry composition (mg/film) |
|---|---|---|
| Maltodextrin | 58.0 | 165.88 |
| Glycerin | 15.0 | 42.90 |
| Mannitol | 5.0 | 14.30 |
| PVP-VA | 2.0 | 5.72 |
| Water | 10.0 | 28.6 |
| Tween 20 | 0.57 | 1.63 |
| Capryol 90 | 0.93 | 2.66 |
| Mint flavor | 1.25 | 3.58 |
| Sucralose | 0.05 | 0.14 |
| Titanium dioxide | 0.20 | 0.57 |
| Tadalafil | 7.00 | 20.0 |
| Total | 100.00 | 286.00 |

Comparison of Dissolution Profiles:

The dissolution profiles of Tadalafil 20 mg Orodispersible film obtained above and the reference product Cialis® 20 mg coated tablets were compared.

Dissolution Condition

| | |
|---|---|
| Samples | n = 12 of reference product |
| | n = 12 of test product |
| Apparatus | Basket apparatus (Ph. Eur. Method 2.9.3, Apparatus 1) |
| Medium | Purified water with 0.5% of SDS |
| Temperature | 37° C. ± 0.5° C. |
| Stirring speed | 150 rpm |
| Medium Volume | 1000 ml |
| Sampling time | 10, 20, 30, 45 and 60 minutes |

Results:

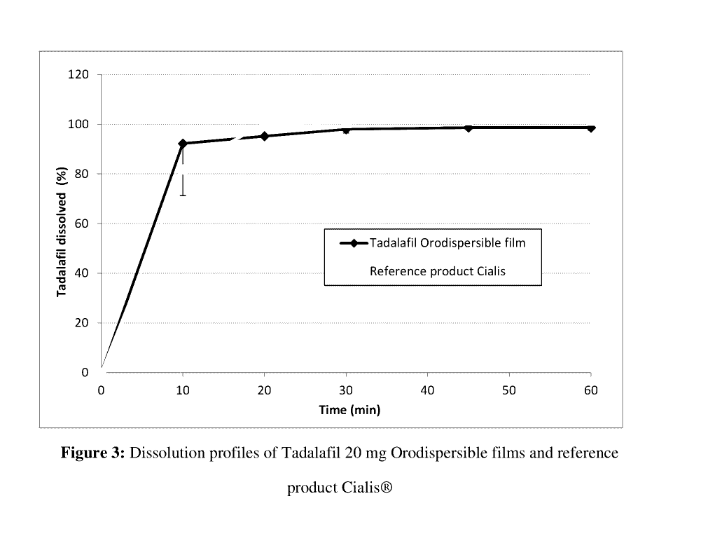

The dissolution profiles of Tadalafil 20 mg Orodispersible film and reference product Cialis® are reported in FIG. 3.

The dissolution profiles of Tadalafil Orodispersible film and reference product are overlapping, suggesting the bioequivalence of the Orodispersible film with the reference product Cialis®.

Moreover disintegration time in the oral cavity of Tadalafil orodispersible film is lower than 3 min, thus the formulation has the same efficacy as the reference drug (tablets) plus the advantages of the orodispersible film.

### Example 7: Progesterone and Testosterone Orodispersible Films Formulations

Orodispersible films containing progesterone and testosterone respectively were prepared according the procedure reported in Example 5, using the components and the amounts reported in the following Tables 14 and 15.

TABLE 14
Composition of Progesterone Orodispersible film

| Compositions | Dry composition w/w % | Dry composition (mg/film) |
|---|---|---|
| Maltodextrin | 41.02 | 189.52 |
| Glycerin | 12.45 | 57.52 |
| Mannitol | 3.00 | 13.86 |
| PVP-VA | 2.00 | 9.24 |
| HP-βCD | 24.66 | 113.94 |
| Water | 7.50 | 34.65 |
| Tween 80 | 0.61 | 2.82 |
| Capryol 90 | 0.89 | 4.11 |
| Blueberry flavor | 5.00 | 23.10 |
| Sucralose | 0.50 | 2.31 |
| Titanium dioxide | 0.18 | 0.83 |
| Colour | 0.02 | 0.09 |
| Progesterone | 7.00 | 10.0 |
| Total | 100.00 | 462.00 |

TABLE 15
Composition of Testosterone Orodispersible film

| Compositions | Dry composition w/w % | Dry composition (mg/film) |
|---|---|---|
| Maltodextrin | 48.65 | 131.35 |
| Glycerin | 15.00 | 40.50 |
| Mannitol | 5.00 | 13.50 |
| PVP-VA | 5.00 | 13.50 |
| HP-βCD | 10.19 | 27.52 |
| Water | 9.00 | 24.30 |
| Tween 80 | 0.57 | 1.50 |
| Capryol 90 | 0.93 | 2.51 |
| Peach flavor | 4.00 | 10.80 |
| Sucralose | 0.50 | 1.35 |
| Titanium dioxide | 0.40 | 1.08 |
| Colour | 0.02 | 0.05 |
| Testosterone | 7.00 | 20.0 |
| Total | 100.00 | 270.00 |

Both orodispersible films containing progesterone and testosterone showed a disintegration time in the mouth lower than 3 minutes.

1. An orodispersible self-supporting film free from hydrocolloids comprising:
a) a film-forming substance consisting of a maltodextrin in an amount between 40 and 80% by weight;
b) one or more plasticizer in a total amount comprised between 15 and 55% by weight;
e) one or more surfactant system in an amount comprised between 0.5 and 6% by weight;
d) an active ingredient for food or therapeutic use in an amount between 0.05 and 30% by weight,
said orodispersible self-supporting film free from hydrocolloids further containing a homopolymer or a copolymer of vinyl acetate in a quantity comprised between 1 and 20% by weight, where the percentages are calculated on the total weight of said film.

2. The orodispersible self-supporting film according to claim 1 wherein the homopolymer or colpolymer of vinyl acetate is polyvinyl acetate or polyvinylpyrrolidone-vinyl acetate.

3. The orodispersible self-supporting film according to claim 2 wherein polyvinyl acetate or polyvinylpyrrolidone-vinyl acetate is present in a quantity comprised between 2 and 10% by weight.

4. The orodispersible self-supporting film according to claim 1 wherein the active ingredient is selected from an inhibitor of type V phosphodiesterase or a hormone.

5. The orodispersible self-supporting film according to claim 4 wherein the active ingredient is an inhibitor of type V phosphodiesterase selected from, sildenafil citrate and tadalafil.

6. The orodispersible self-supporting film according to claim 4 wherein the active ingredient is a hormone selected from progesterone and testosterone.

7. The orodispersible self-supporting film according to claim 1, wherein the vinylacetate homopolymer or copolymer is contained in an amount comprised between 2 and 10% by weight based on the film total weight.

8. The orodispersible self-supporting film according to claim 1, wherein the maltodextrin has a dextrose content, expressed in equivalents, of less than 50.

9. The orodispersible self-supporting film according to claim 8, wherein the dextrose content is between 5 and 40.

10. The orodispersible self-supporting film according to claim 1, wherein the plasticizer is selected from the class consisting of water, polyalcohols, citric acid esters, sebacic acid esters or mixtures thereof.

11. The orodispersible self-supporting film according to claim 10, wherein the plasticizer is selected from the class consisting of water, propylene glycol, glycerine, sorbitol, maltitol and mixtures thereof.

12. The orodispersible self-supporting film according to claim 1, wherein the surfactant system consists of one or more surfactants.

13. The orodispersible self-supporting film according to claim 12, wherein said surfactants are selected from the group consisting of sorbitan derivatives, sorbitol derivatives, esters of sucrose, fatty acid esters and mixtures thereof.

13. A process for the preparation of self-supporting film according to claim 1, comprising the following steps:
i) dispersing the maltodextrin, the plasticizer, the surfactant system, the homopolymer or copolymer of vinyl acetate and the active ingredient for therapeutic or food use in a polar solvent;
ii) laminating the mixture obtained in the previous step on a release liner;
iii) drying in the oven at a temperature preferably comprised between 50 and 140° C.;
iv) removing the release liner from the film obtained in the preceding step;
v) cutting the film with the desired dimensions and packaging them.
